(19) United States
(12) Patent Application Publication
(10) Pub. No.: US 2007/0264312 A1
(43) Pub. Date: Nov. 15, 2007

(54) Rheologically Modified Edible Oils

(76) Inventors: Crawford Bryan Skaggs, San Diego, CA (US); Puspendu Deo, San Diego, CA (US); Todd Talashek, San Diego, CA (US)

Correspondence Address: JANE SHERSHENOVICH, 1000 PARKWOOD CIRCLE, SUITE 1000, ATLANTA, GA, 30339, US

(21) Appl. No.: 11/382,979

(22) Filed: May 12, 2006

(51) Int. Cl. A23L 1/222; A61K 47/00

(52) U.S. Cl. 424/439; 426/651

(57) ABSTRACT

The invention is an improved form of a rheologically modified fluid which is capable of suspending various particulates including polysaccharides, hydrocolloids, and other food approved items, in a pumpable oil-based carrier fluid. The modified fluid can be thickened such that it can be used as a lubricant or a food item. The modified fluid preferably contains food approved ingredients. A representative system comprises a carrier fluid, a thickening or gelling hydrocolloid, and an oil-thickening compound. A preferred carrier fluid is vegetable oil, a preferred hydrocolloid is xanthan gum, and a preferred oil thickening compound is fumed silica.

### BACKGROUND OF THE INVENTION

Edible oils are used in various food products and during the processing of these products. It is highly desired within the food industry to modify the rheology of edible oils for their expanded use in the food industry. Edible oils are intended for human consumption and include oils used in animal feeds where the animal is intended for human consumption.

Particulated solids, including hydrocolloids such as xanthan gum, and/or other food approved stabilizers are typically used in food products to control water. Other particulated materials, such as proteins, spices, colorants, etc. are routinely added to food systems for a variety of reasons, including to boost the nutritional profile (increased protein), to improve marketability (color), to improve texture or mouthfeel, or to impart a favorable taste (spices).

Hydrocolloids, xanthan gum for example, are frequently used to thicken and stabilize fluid food systems, such as sauces, marinades, salad dressings, pourable dressings, spoonable dressings, beverages, whipped toppings, low fat margarines, low fat vegetable oil spreads, low fat mayonnaise, meat brines, and others that would be known in the art. In order for the hydrocolloids to work effectively as thickeners and stabilizers, the hydrocolloid must first be hydrated in these food systems. Because certain hydrocolloids are very effective at thickening water-based systems, only a small amount is required (typically less than 0.5% by weight). To maintain microbial stability, hydrocolloids are typically sold to manufactures in a dry powdered form. For example, xanthan gum is currently sold to food manufacturers in a dry (about 90% solids) powdered form.

However, particulates, xanthan is one example, in the powdered form have several disadvantages for food processors. The food processor must first hydrate the powdered particulate for it to function successfully in the food application. Certain hydrocolloids, for example xanthan, are high molecular weight polysaccharides that hydrate slowly in water and require extensive mixing equipment and mixing time.

Moreover, hydrocolloids, such as xanthan gum, are prone to forming unhydrated lumps if not dispersed properly, so additional steps must be taken to ensure proper dispersion of the product. This may involve additional processing steps such as dispersing the hydrocolloid in other powdered or non-aqueous additives prior to the addition of water thereby extending the food processing time. The nature of the hydrocolloid powders can also pose difficulties for the food processor. Hydrocolloid samples can contain ‘fines’ or some very small hydrocolloid particulates as a result of the milling process. These fines readily become airborne, thus causing safety and environmental issues for the food processor.

The present invention, a rheologically modified carrier fluid, remedies many of the handling and performance disadvantages associated with the dry, powdered form of particulates. Using the carrier fluid of the current invention also avoids the safety or environmental issues with airborne fine particulates because the particulates remain suspended in the carrier fluid. A significant advantage of this invention is that it enables food manufacturers to use computer aided process control to add and meter the ingredients. This improves quality and can reduce labor costs.

Liquid concentrates, such as for xanthan, have been used as an alternative to dry powders in the past. However, transportation of liquid concentrates has proven to be cost prohibitive due to high transportation costs. These prior liquid concentrates typically contained relatively low levels of hydrocolloid, averaging ˜1-10% hydrocolloid by weight (˜5% for xanthan). The high loading levels of particulates (≧10-45% by weight) uniformly suspended in the carrier fluid of the present invention make the carrier fluid system more economical for transportation. For certain embodiments, the concentration is increased to about 75% by weight. Furthermore, with the particulate suspended in the liquid phase, it is already fully dispersed so there will be no dispersion issues for the food processor.

The fluid nature of the carrier fluid described in the current invention will allow food manufacturers to pump particulates into the formulation of liquid food systems, which allows for metering the proper particulate concentration based on volume.

Certain fluidized water-soluble hydrocolloid dispersions have been attempted in the past but have all failed to a certain degree to provide overall effective results, particularly within the food industry. For instance, xanthan concentrates contain more than 90% water and are therefore prohibitively expensive to ship. In addition, it is difficult to maintain long-term microbiological stability in a water based concentrate. Thus, other non-aqueous solvent systems were required. The existence of certain of these vehicles prohibited the end product from being incorporated into food applications.

U.S. Pat. No. 5,096,490 describes a fluid suspension of CMC for paper coating applications. U.S. Pat. No. 6,825,248 describes a mineral oil-based fluidized polymer suspension composition for use as a rheology modifier in paper coatings. These, among other liquid slurry systems described in the past, are limited because they utilize ingredients that are not approved for use in food. With the present invention, all ingredients, including the oil-thickening fumed silica, have been approved for use in food systems.

Fluidized polymer suspensions using organoclay and water-soluble polymer have been previously described. WO 2005/116114A1 and US 2005/0256232 describe a nonaqueous fluidized polymer suspension containing at least one water-soluble polymer, a low molecular weight polyethylene glycol (PEG), an optional dissolution additive, and at least one organoclay suspending aid to permit effective long-term, uniform, storage-stable fluidizing of the polymer for use in paper and paint applications. A drawback of these systems is also the requirement for additives such as organoclays which are not approved for use as food ingredients.

### BRIEF SUMMARY OF THE INVENTION

The present invention is directed to rheologically modified edible oils. Examples of these edible oils include but are not limited to sunflower oil, canola oil, flax seed oil, soybean oil, almond oil, peanut oil, grape seed oil, rice oil, palm oil, medium chain triglycerides, and coconut oil. Other acceptable oils will be readily apparent to those skilled in the art. The oils of the present invention possess improved rheology for the uses described herein.

The present invention is also directed to compositions of matter wherein edible oils are viscosified by blending with fumed silica and water. When the fumed silica is about 1 to about 5% basis total weight, the edible oil rheology is sufficient to suspend particulates such as xanthan gum, but flows readily and is easy to mix, pump, and convey.

At higher silica loading, the edible oil becomes very thick and could provide value as a machine lubricant in food applications. The thicker edible oil can also be used for making a coating that could be brushed onto a grill or other cooking surface. Healthy spreads can also be developed using this technology. For example, an olive oil can be viscosified and used as a spread or a component in other foods. Flavored oils may also be included in spreads to improve the taste.

Particulated solids, including hydrocolloids such as xanthan gum as one non-limiting example, and/or other food approved stabilizers are typically used in food products to control water. Other particulated solids, such as proteins, spices and flavorings, colorants, etc. are routinely added to food systems for a variety of reasons including to boost the nutritional profile (increased protein), to improve marketability (color), or to impart a favorable taste (spices). A comprehensive listing of available food additives is included in the Food Chemical Codex, 5^{th} Edition, 1993.

Hydrocolloids, xanthan gum is one non-limiting example, are frequently used to thicken and stabilize fluid foods, such as sauces, marinades, salad dressings, pourable dressings, spoonable dressings, beverages, whipped toppings, low fat margarines, low fat vegetable oil spreads, low fat mayonnaise, meat brines, and others that would be known in the art. In order for the hydrocolloids to work effectively as thickeners and stabilizers, the hydrocolloid must first be hydrated in these food systems. Because certain hydrocolloids such as xanthan are very effective at thickening water-based systems, only a small amount is required (typically less than 0.5% by weight). For most hydrocolloids, concentrations greater than 5% by weight renders the solutions very viscous and gel-like, which make them difficult to produce and transport. CMC, among other hydrocolloids known to those skilled in the art, is an exception, having a low viscosity form which even at a 5% solution wouldn't be difficult to make or pour. However, rendering these aqueous concentrates stable to microbial growth is problematic. As a result, hydrocolloids are sold to manufactures in a dry powdered form. For example, xanthan gum is currently sold to food manufacturers in a dry (about 90% solids), powdered form. This reduces the cost associated with shipping a large quantity of water that would be present in a liquid hydrocolloid concentrate.

The present invention, a rheologically modified carrier fluid, remedies many of the handling and performance disadvantages associated with the dry, powdered form of particulates. Using the carrier fluid of the current invention also avoids the safety or environmental issues with airborne fine particulates because the particulates remain suspended in the carrier fluid.

Existing liquid concentrates average ˜1-10% hydrocolloid by weight (˜5% for xanthan). The high loading levels of particulates (≧10-45% by weight) in the carrier fluid of the present invention (20-45% by weight for xanthan) make the carrier fluid system more economical for transportation. Certain embodiments contain particulates at about 75% by weight. With the particulate suspended in the liquid phase, it is already fully dispersed so there will be no dispersion issues for the food processor. The ability to provide such a proper nonaqueous water-soluble polysaccharides (xanthan, for instance) dispersion has not been forthcoming within the pertinent art.

The present invention is directed to compositions comprising a rheologically modified edible oil or edible oils with flavorings.

The present invention is further directed to compositions comprising a Theologically modified edible oil suitable for use as a coating for cooking utensils, pots, pans, grills, and other surfaces that contact food.

The present invention is further directed to compositions comprising a Theologically modified edible oil suitable for use as a lubricant.

The present invention is further directed to compositions comprising a rheologically modified carrier fluid for particulates.

The present invention is further directed to methods for making the compositions described herein.

The present invention is further directed to methods of making food applications by metering in a fluidized form of hydrocolloids.

The present invention is further directed to use of the carrier fluids in food systems.

The present invention is further directed to food systems containing the carrier fluid.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 depicts xanthan slurries immediately after mixing

FIG. 2 depicts xanthan slurries after 24 hour of storage at ambient temperature

FIG. 3 depicts xanthan slurries after one week of storage at ambient temperature.

### DETAILED DESCRIPTION OF THE INVENTION

A rheologically modified edible oil was developed. CAB-O-SIL® M-5 fumed silica from Cabot Corporation was used to thicken vegetable oil. The greatest thickening efficiency with the fumed silica is realized when —OH groups on the silica surface can bond to each other to form network structure. The thickening of the oil more importantly depends on the silica concentration, the amount of water and the amount of surfactant present in the system. The silica concentrations used for thickening the oil are in the range of about 1% to about 5%. The amount of surfactants (mixtures of Span 80 and Tween 80) added to thicken the oil are in the range of about 0.0% to about 1%. The amount of water added is about 0% to about 1%. The impact of the addition of fumed silica, fumed silica+water, and fumed silica+surfactant on the behavior of vegetable oil is shown in Table 1. All values presented herein are on a weight basis unless otherwise noted. The percentage of fumed silica, water and surfactant are on the basis of total solution weight (fumed silica, fumed silica+water, fumed silica+surfactant). All data presented here are at ambient temperature unless otherwise noted.

One approach to produce a liquid delivery system for xanthan gum is to activate the fumed silica with the appropriate amount of water to form hydrogen bonds among themselves in vegetable oil at lower loading. Such examples are shown in Table 1. As the silica is activated, the silica particles can come together and form a rigid network. Oil is trapped in the silica network, resulting in increased viscosity of the system. With higher concentrations of silica, there is an increase in the viscosity of the system, whereas with higher amounts of water, the system is destabilized. Limiting the water concentration minimizes the solution viscosity.

A second approach for producing a liquid delivery system for xanthan gum is to add a mixture of surface active agents (surfactants) capable of interacting with the silica particles in vegetable oil. As the hydrophilic surfactant head groups interact with the silica particles, the hydrophobic tails will interact among themselves. This interaction forms a network that traps the oil and results in increased viscosity of the system. Such examples are shown in Table 1. With higher concentrations of silica and surfactants, there is an increase in the viscosity of the system. Limiting the surfactant concentration minimizes the solution viscosity.

A third approach for producing a liquid delivery system for xanthan gum is to add a higher amount of fumed silica to the vegetable oil. At higher levels of silica, the silica particles can effectively form a silica network in the system. Such examples are shown in Table 1. With higher concentrations of silica, there is an increase in the viscosity of the system. Limiting the silica concentration minimizes the solution viscosity.

The present invention is directed to compositions of matter where edible oils are viscosified by blending with fumed silica and water (about 0.2% basis total volume.). Depending on the application, water may or may not be required but is tolerated in the fluid. The physical properties of this rheologically modified oil are related to the fumed silica concentration. When the fumed silica is about 1 to about 5% basis total weight, the edible oil rheology is sufficient to suspend particulates such as xanthan gum. However, this suspension readily flows making it easy to mix, pump, and convey.

At higher silica loading, the edible oil becomes very thick and could provide value as a machine lubricant in food applications. The thicker edible oil can also be used for making coatings that could be brushed onto a grill or other cooking surface. Healthy spreads can also be developed using this technology. For example, an olive oil can be viscosified and used as a spread or a component in other foods and this component may contain flavorings.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention.

All parts, percentages and ratios used herein are expressed by weight unless otherwise specified. All documents cited herein are incorporated by reference.

### EXAMPLE 1

To 489 g of vegetable oil, 10 g of CAB-O-SIL® M-5 fumed silica is added and dispersed throughout the oil with mixing. 1 g of water is then added. The vegetable oil, fumed silica, and water are then mixed for 5 minutes on a Silverson mixer at 6,000 rpm to thicken the oil. The fumed silica-thickened vegetable oil has sufficient rheology to suspend xanthan gum particles. This suspension readily flows and is easy to mix, pump, and convey. Methods used to measure mixing, pumping, and conveyance of materials are well known to those skilled in the art. The shear viscosity at high shear rates (≧20 sec⁻¹) typically predicts the flow characteristics of the fluid during pumping or mixing. See Table 1.

### EXAMPLE 2

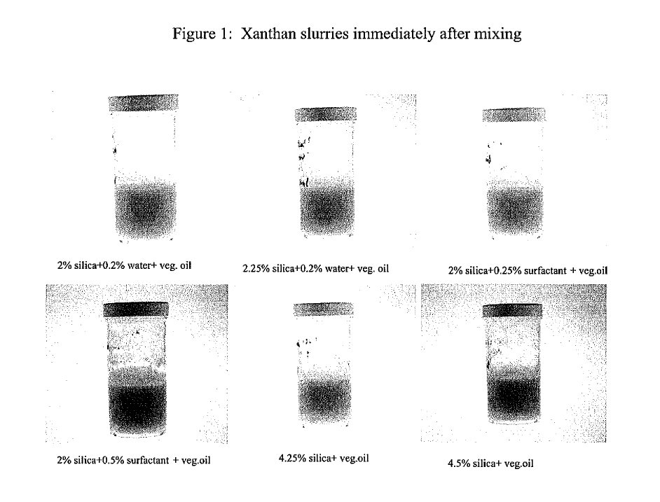

The xanthan slurry is prepared using the above mentioned thickened vegetable oils. The desired amount of the xanthan gum is added to the modified oils to prepare the slurry. The concentration of the gum here is 40%. After the addition of the gum, the slurries are stirred for 20 minutes using a bench top mixer. The nature of the slurry (free flowing or thick paste) depends on the amount of fumed silica or fumed silica+water or fumed silica+surfactant present in the system. The stability of the slurries at ambient temperature is tested by monitoring them at different intervals of time. FIG. 1 shows the slurry just after mixing. From this figure, it can be seen that all the slurries are stable.

### EXAMPLE 3

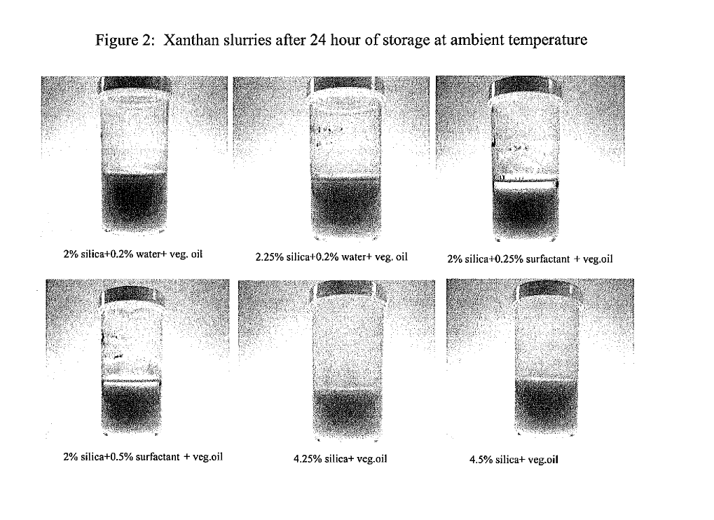

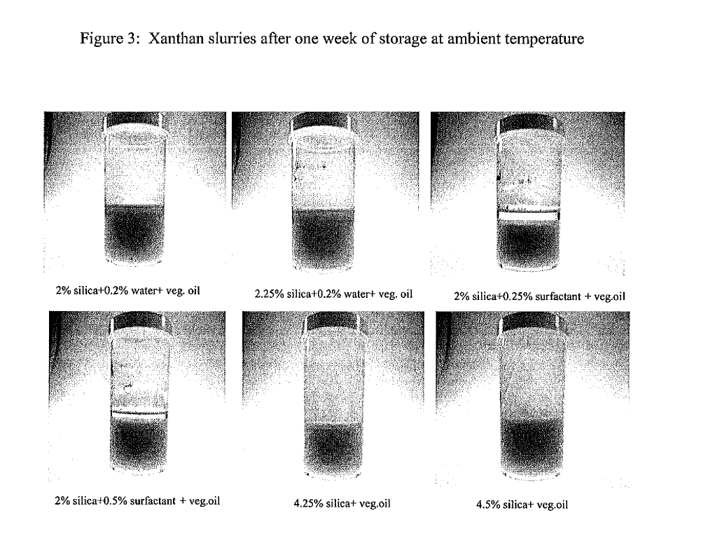

After 24 hours of storage at ambient temperature, there is top layer separation of the oil in the system containing fumed silica+water and fumed silica+surfactant, which can be seen in FIG. 2. The system containing fumed silica without water and surfactant is quite stable and there is no separation at all. FIG. 3 shows the stability of the above systems after a week of preparation. This figure clearly shows that the slurries prepared with the system containing only fumed silica and vegetable oil are quite stable after a week of storage at ambient temperature. Clear separation of the oil layer at the top can be observed for the systems containing fumed silica+water and fumed silica+surfactant. This is because water and surfactant help fumed silica particles to form aggregates. So at lower concentrations of silica, water or surfactant can bring the silica particles together to form aggregates. The formation of these networks helps to trap the oil, hence modifying the viscosity of the oil system. But the addition of xanthan gum to the system disturbs the network by either interacting or taking away the water or surfactant from the silica, hence breaking the network. With the breaking of these networks, more and more oil trapped in the network comes out at the top of the slurry. Whereas, in the case of silica only, the network among the silica particles is much stronger than the network formed with the help of water and surfactant. So the addition of xanthan gum has little effect on the system containing fumed silica and vegetable oil.

TABLE 1
Impact of the addition of fumed silica, fumed silica + water
and fumed silica + surfactant on vegetable oil viscosity.

| | Viscosity@ | Viscosity@ | Viscosity@ | Viscosity@ | Viscosity@ |
|---|---|---|---|---|---|
| Sample | 20 S⁻¹ | 7 S⁻¹ | 1 S⁻¹ | 0.1 S⁻¹ | 0.01 S⁻¹ |
| Vegetable oil | 52 | 54 | * | * | * |
| 2% Silica + | ** | ** | ** | 70,480 | 415,200 |
| 0.2% Water + | | | | | |
| Veg. oil | | | | | |
| 2.25% Silica + | ** | ** | ** | ** | 700,000 |
| 0.2% Water + | | | | | |
| Vegetable oil | | | | | |
| 2.5% Silica + | ** | ** | ** | ** | ** |
| 0.2% Water + | | | | | |
| Vegetable oil | | | | | |
| 2% Silica + | ** | ** | 7,352 | 58,480 | 350,000 |
| 0.25% Surfactant + | | | | | |
| Vegetable oil | | | | | |
| 2% Silica + | ** | ** | ** | 68,960 | 434,300 |
| 0.5% Surfactant + | | | | | |
| Vegetable oil | | | | | |
| 3% Silica + | 114 | 218 | 465 | 2500 | 11,900 |
| Vegetable oil | | | | | |
| 4% Silica + | 214 | 328 | 784 | 4,000 | 27,200 |
| Vegetable oil | | | | | |
| 4.25% Silica + | 367 | 650 | 1,832 | 10,880 | 73,600 |
| Vegetable oil | | | | | |
| 4.5% Silica + | 441 | 774 | 2,184 | 12,880 | 89,600 |
| Vegetable oil | | | | | |
| 5% Silica + | ** | 1,050 | 4,500 | 21,400 | 125,500 |
| Vegetable oil | | | | | |

Viscosity expressed as m Pa · s (1 m Pa · s = 1 cP)
% water, surfactant and silica are percentage of these materials added basis of total solution weight.
* = Below minimum torque required for accurate measurement
** = Maximum torque exceeded

### EXAMPLE 4

A creamy, smooth textured Italian dressing was prepared with a xanthan gum slurry. The dressing was easily pourable and eye appealing. The dressing had excellent emulsion stability, flavor release, and mouth feel.

The slurry was hydrated with available water under vigorous agitation conditions for 15 minutes. A dry blend of the remaining ingredients was added to the slurry. Egg yolks were then introduced into the slurry, followed by vegetable oil, vinegar, and lemon juice. The mixture was homogenized using a Colloid Mill, with a mill setting of 0.25 mm (00.1 in.). The ingredient formulation is presented in Table 2.

TABLE 2
Salad dressing using a rheologically modified oil.

| | QUANTITY | |
|---|---|---|
| INGREDIENTS | GRAMS | PERCENT |
| Vegetable oil | 550.0 | 54.84 |
| Water | 209.0 | 20.84 |
| Cider vinegar, 5% (50 grain) | 150.0 | 14.96 |
| Lemon juice, single strength | 30.0 | 2.99 |
| Egg yolks, frozen, salted | 20.0 | 1.99 |
| Sugar, granular | 15.0 | 1.50 |
| Salt | 10.0 | 1.00 |
| Monosodium glutamate | 5.0 | 0.50 |
| Garlic powder | 4.0 | 0.40 |
| Onion powder | 3.0 | 0.30 |
| Oregano powder | 2.0 | 0.20 |
| Xanthan gum slurry (2.6% fumed silica, | 5.0 | 0.50 |
| 40% xanthan, 57.4% vegetable oil). | | |
| TOTAL | 1003 g | 100.0% |

I/We claim:

1. A rheologically modified fluid comprising about 1% to about 10% fumed silica and about 90% to about 99% edible oil.

2. The rheologically modified fluid of claim 1 wherein the fumed silica is present in an amount from about 2% to about 5% by weight.

3. The rheologically modified fluid of claim 1 wherein the fumed silica is present at about 4.25% by weight.

4. The rheologically modified fluid of claim 1 further comprising a particulate.

5. The rheologically modified fluid of claim 4 wherein said particulate is present at about 20 to about 45%.

6. The rheologically modified fluid of claim 4 wherein said particulate is present at about 30 to about 45%.

7. The rheologically modified fluid of claim 4 wherein said particulate is present at about 40%.

8. The rheologically modified fluid of any of claims 1-7 further comprising water.

9. The rheologically modified fluid of any of claims 1-8 further comprising a surfactant.

10. The rheologically modified fluid of claim 4 wherein said particulate is selected from the group consisting of polysaccharides, proteins, minerals, colorants, spices, and hydrocolloids.

11. The rheologically modified fluid of claim 10 wherein said hydrocolloid is xanthan.

12. A food product comprising the rheologically modified fluid of any of claims 1-11.

13. Method of making the composition of any of claims 1-12.

14. The food item of claim 12 wherein said food item is a salad dressing.

15. Method of making a salad dressing whereby a dose of a rheologically modified fluid is introduced into the manufacturing process wherein ingredients are metered into the salad dressing as a fluid.

16. Use of the rheologically modified fluid composition of any of claims 1-3, 8 or 9 as a coating for surfaces that contact food.

17. Use of the rheologically modified fluid composition of any of claims 1-3, 8 or 9 as an edible lubricant.

18. The rheologically modified fluid of claim 4 wherein said particulate is present at about 45 to about 75%.
